(19) United States
(12) Patent Application Publication
(10) Pub. No.: US 2007/0207948 A1
(43) Pub. Date: Sep. 6, 2007

(54) Convergent Synthesis for Kahalalide Compounds

(76) Inventors: Andres Francesch Solloso, Madrid (ES); Angel Lopez Macia, Madrid (ES); Carmen Cuevas Marchante, Madrid (ES); Fernando Albericio Palomera, Barcelona (ES); Gerardo Acosta Crespo, Barcelona (ES); Luis Javier Cruz Ricondo, Barcelona (ES); Ernest Giralt Lledo, Barcelona (ES); Carolina Gracia Cantador, Barcelona (ES); Albert Isidro Lobet, Barcelona (ES)

Correspondence Address: KING & SPALDING, 1185 AVENUE OF THE AMERICAS, NEW YORK, NY, 10036-4003, US

(21) Appl. No.: 11/587,177

(22) PCT Filed: Apr. 22, 2005
(86) PCT No.: PCT/GB05/01537

§ 371 Date: Jan. 2, 2007

(30) Foreign Application Priority Data
Apr. 22, 2004 (GB) 0408958.7

(51) Int. Cl. A61K 38/00

(52) U.S. Cl. 514/9

(57) ABSTRACT

New synthetic routes to kahalalide compounds are provided. These are based on convergent approaches using orthogonal protecting schemes, where a better control of the intermediates is taken. Kahalalide F or a mimic of kahalalide F is synthesised by coupling a cyclic part with a side chain fragment, e.g. according to the reaction (1)

### FIELD OF THE INVENTION

The present invention is directed to new synthetic routes for kahalalide compounds and related compounds.

### BACKGROUND OF THE INVENTION

The kahalalide compounds are peptides isolated from a Hawaiian herbivorous marine species of mollusc, Elysia rufescens and its diet, the green alga Bryopsis sp. Kahalalide F is described in Hamann et al., J. Am. Chem. Soc., 1993, 115, 5825-5826.

Kahalalide A-G are described in Hamann, M. et al., J. Org. Chem, 1996, 61, 6594-6600: “Kahalalides: bioactive peptides from a marine mollusk Elysia rufescens and its algal diet Bryopsis sp.” Kahalalide F has the following structure:

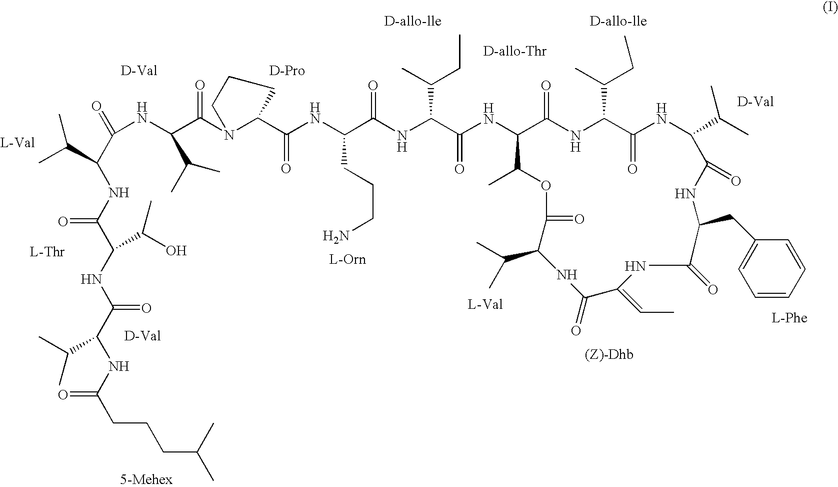

Kahalalide H and J are described in Scheuer P. J. et al., J. Nat. Prod. 1997, 60, 562-567: “Two acyclic kahalalides from the sacoglossan mollusk Elysia nufescens”.

Kahalalide O is described in Scheuer P. J. et al., J. Nat. Prod. 2000, 63(1) 152-4: “A new depsipeptide from the sacoglossan mollusk Elysia ornata and the green alga Bryopsis species”.

For kahalalide K, see Kan, Y. et al., J. Nat. Prod. 1999 62(8) 1169-72: “Kahalalide K: A new cyclic depsipeptide from the Hawaiian green alga bryopsis species”.

For related reports, see also Goetz et al., Tetrahedron, 1999, 55; 7739-7746: “The absolute stereochemistry of Kahalalide F”; Albericio, F. et al. Tetrahedron Letters, 2000, 41, 9765-9769: “Kahalalide B. Synthesis of a natural cyclodepsipeptide”; Becerro et al. J. Chem. Ecol. 2001, 27(11), 2287-99: “Chemical defenses of the sarcoglossan mollusk Elysia rufescens and its host Alga bryopsis sp.”.

Of the kahalalide compounds, kahalalide F and analogues (Formula 1 below) are the most promising because of antitumoral activities. The structure is complex, comprising six amino acids as a cyclic part, and an exocyclic chain of seven amino acids with a terminal aliphatic/fatty acid group.

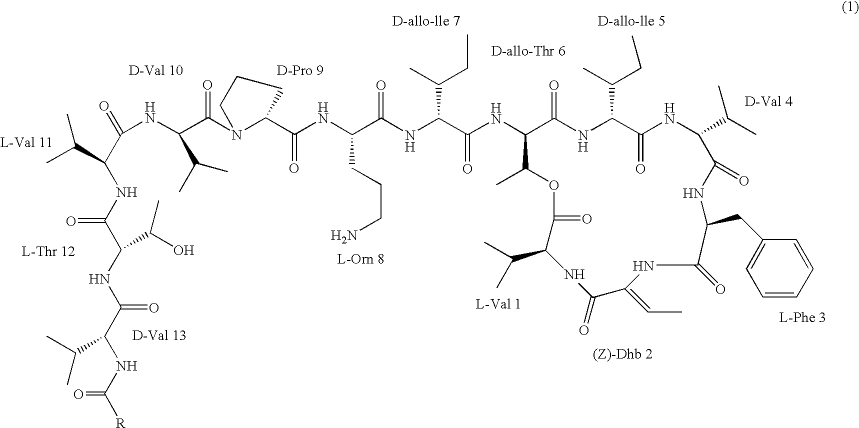

Among those of more interesting activity is when R is a 5-methylhexanoyl or the isomer where R is a 4(S)-methylhexanoyl group.

The activity of kahalalide F against in vitro cell cultures of human lung carcinoma A-549 and human colon carcinoma HT-29 were reported in EP 610 078. Kahalalide F has also demonstrated to have antiviral and antifungal properties, as well as to be useful in the treatment of psoriasis.

WO 02 36145 describes pharmaceutical compositions containing kahalalide F and new uses of this compound in cancer therapy and is incorporated herein by reference its entirety.

WO 03 33012 describes the clinical use in oncology of kahalalide compounds and is incorporated herein by specific reference in its entirety.

The synthesis and cytotoxic activities of natural and synthetic kahalalide compounds is described in WO 01 58934, which is incorporated herein by specific reference in its entirety. WO 01 58934 describes the synthesis of kahalalide F and also of mimic compounds with a similar structure in which amino acids are replaced by other amino acids or the terminal fatty acid chain is replaced by other fatty acids. In particular, it describes a solid phase synthesis of kahalalide F (I) and derivatives and analogues, in accordance with the following scheme: 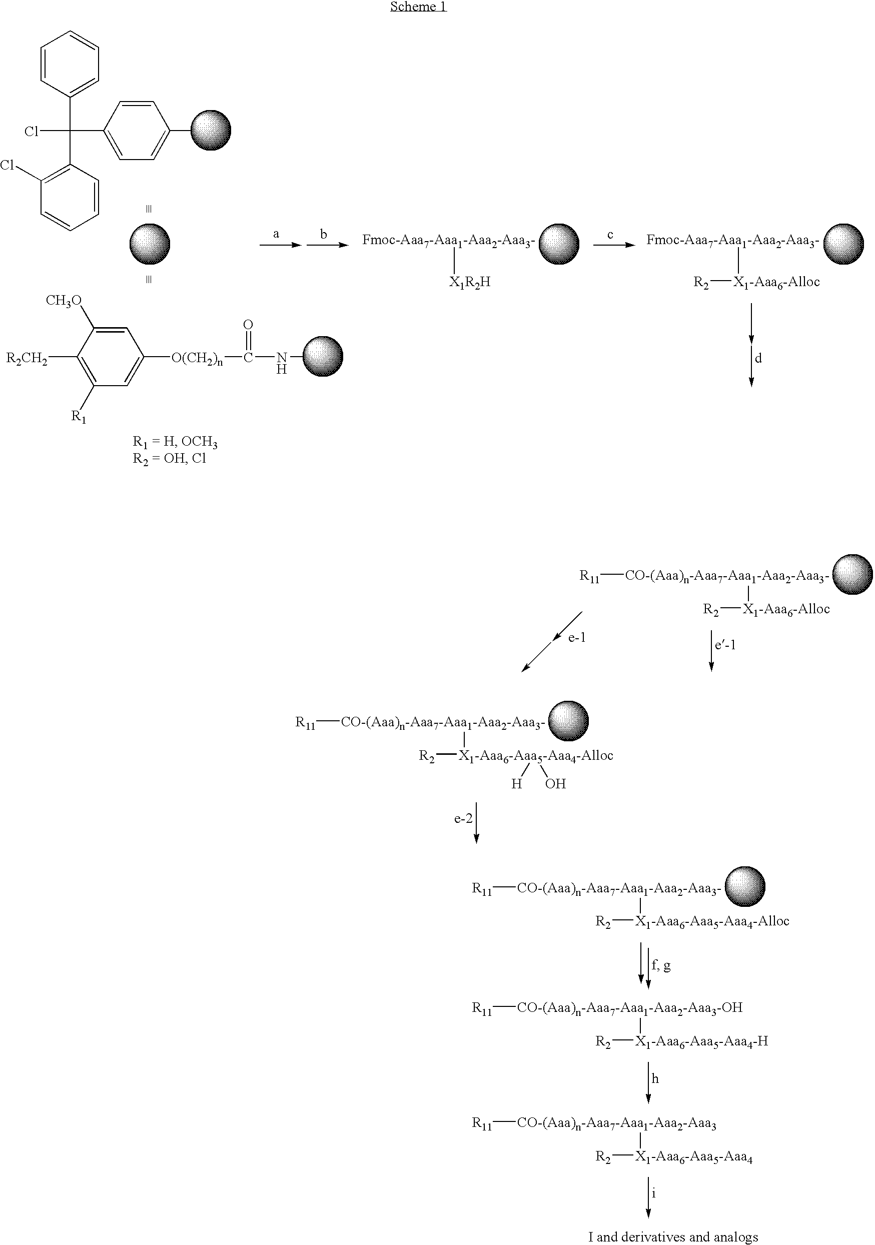
where the various groups take the meanings given in WO 01 58934. Solid phase synthesis is employed to generate a partially protected open chain compound, which is then cleaved, cyclized and deprotected.

WO 2004035613 relates to the 4(S)-methylhexanoyl isomer mentioned above and other compounds.

WO 2005023846 describes the synthesis of more mimic compounds with a similar structure of kahalalide F in which amino acids are replaced by other amino acids or the terminal fatty acid chain is replaced by other aliphatic/fatty acids. It uses the synthetic route of WO 01 58934. WO 2005023846 is incorporated herein by specific reference in its entirety.

There is still a need to provide synthetic routes for kahalalide compounds.

### SUMMARY OF THE INVENTION

We have found several new improved routes for the preparation of kahalalide analogues. The previous strategy was based in a stepwise solid-phase synthesis of the partial protected open chain of the kahalalide, followed by the cyclization carried out in solution, and finally, a removal of the protecting groups in solution as well.

The new routes are based in convergent approaches, where a better control of the intermediates is taken, with more reactions carried out in solution, and therefore with more characterization of the intermediates.

The invention is also directed to a process for the preparation of new analogues of parent compounds.

Thus, the present invention provides a synthetic route to natural kahalalides, especially kahalalide F, and mimics of natural kahalalides. The mimic compounds may differ in one or more amino acids, and/or one or more components of the acyl side chain.

Suitably the mimics of this invention have at least one of the following features to differentiate from a parent naturally occurring kahalalide:

1 to 7, especially 1 to 3, more especially 1 or 2, most especially 1, amino acid which is not the same as an amino acid of the parent compound;

1 to 10, especially 1 to 6, more especially 1 to 3, most especially 1 or 2, additional methylene groups in the side chain acyl group of the parent compound;

1 to 10, especially 1 to 6, more especially 1 to 3, most especially 1 pr 2, methylene groups omitted from the side chain acyl group of the parent compound;

1 to 6, especially 1 to 3, more especially 1 or 3, substituents added to or omitted from the side chain acyl group of the parent compound;

omission of the 5-methyl substituent from the acyl group of the side chain; and

omission of the acyl group of the side chain.

In particular, the mimic is preferably a kahalalide F derivative which is not a mix of isomers known as kahalalide F. Such a derivative can have a structure with a cyclic part and a side chain derived from the formula (I): 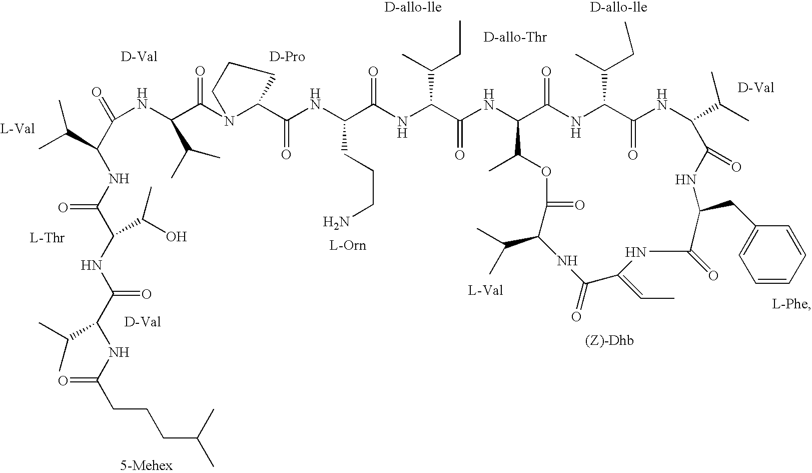
the derivative differing from the formula (I) in one or more of the following respects:

1 or 2 amino acids which are not the same as an amino acid in the structure of formula (I);

1 to 10 additional methylene groups in the acyl group of the side chain of the structure of formula (I);

1 to 5 methylene groups omitted from the acyl group of the side chain of the structure of formula (I);

1 to 3 substituents added to the side chain acyl group of the structure of formula (I);

omission of the 5-methyl substituent from the acyl group of the side chain; and

omission of the acyl group of the side chain.

For example, the compound can differ from the formula (I) in one or more of the following respects:

1 amino acid which is not the same as an amino acid in the structure of formula (I);

1 additional methylene group in the side chain acyl group of the structure of formula (I);

1 methylene group omitted from the side chain acyl group of the structure of formula (I);

1 substituent added to the side chain acyl group of the structure of formula (I);

omission of the 5-methyl substituent from the acyl group of the side chain.

The 1 or 2 amino acids which are not the same as an amino acid in the structure of formula (I) can be omitted amino acids. There can be omission from the cyclic part of the structure.

In one aspect, each amino acid is as in formula (I). The side chain can be a congener of 5-MeHex-D-Val-L-Thr-L-Val-D-Val-D-Pro-L-Orn-D-allo-Ile. For instance, the 5-MeHex can be replaced by a terminal alkyl, alkenyl, alkynyl, aryl, aralkyl, heteroaryl, heteroalkyl, or alicyclic group, especially a terminal alkyl group. Such a group can have 4 to 10 carbon atoms. The terminal alkyl group suitably has 1 or more methyl or ethyl groups branching distal to the point of attachment to the rest of the molecule, and preferably a single branched methyl group.

The terminal alkyl group can be substituted with one or more halogen, hydroxy, alkoxy, amino, carboxyl, carboxamido, cyano, nitro, alkylsulfonyl, alkoxy, alkoxyalkyl, arylalkylaryl, heterocyclic, alicyclic, aryl or aralkyl groups.

Chirality can be present in the replacement terminal group, and the invention embraces the individual isomers as well as mixes thereof including racemic mixes.

More details of preferred definitions and typical compounds are given in the texts incorporated herein by reference, WO 01 58934 and WO 2005023846. We currently prefer that the terminal acyl group in the sidechain is 5-methylhexanoyl, 4-methylhexanoyl or more especially 4(S)-methylhexanoyl. Especially preferred is a mimic which is of the formula given above for kahalalide F but has a 4(S)-methylhexanoyl group.

The process of this invention involves coupling a cyclic part with a side chain fragment. The cyclic part can itself contain at least one of the side chain amino acids. Alternatively the side chain fragment can correspond to the complete side chain of the desired compound.

The cyclic part is preferably that of kahalalide F or of a mimic defined above, and can contain one or more amino acids that are present in the side chain of the desired compound. It is preferred that the direct product of the coupling is an optionally protected form of the desired end product, kahalalide F or a mimic. Thus the preferred reactions consist of the coupling step and then deprotection to give the desired compound.

### DETAILED DESCRIPTION OF THE INVENTION

We have identified new synthetic routes to kahalalide compounds. These are based in convergent approaches using orthogonal protecting schemes, where a better control of the intermediates is taken.

Convergent strategies are defined as those in which peptide fragments are coupled together to give the desired target molecule. The condensation of peptide fragments should lead to fewer problems in the isolation and purification of intermediates. The difference between the desired condensation product and the segments themselves, in terms of molecular size and chemical nature, should be sufficiently pronounced so as to permit their separation relatively easily (Lloyd-Williams, P.; Albericio, F.; Giralt, R. “Chemical approaches to the synthesis of peptides and proteins”. CRC Press. Boca Raton (FL), 1997).

An orthogonal protecting scheme has been defined as one based on completely different classes of protecting groups such that each class of groups can be removed in any order and in the presence of all other classes of protecting group (Barany, G.; Albericio, F. J. Am. Chem. Soc, 107, 4936 (1985)).

Preparation of protected peptides can be carried out in solution and/or in solid-phase, as well as from natural kahalalide F isolated from either the mollusc or the alga or obtained by fermentation. Assembling of the protected peptides are preferably carried out in solution. All intermediates can be characterized and, if needed, purified.

Examples of strategies covered by this invention are shown in the following Scheme I: 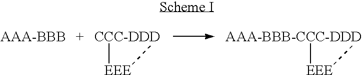
wherein AAA is an aliphatic/fatty acid or an aliphatic/fatty acylamino acid or a peptide and BBB, CCC, DDD and EEE are amino acids or peptides. Amino acids are independently selected from natural or non-natural amino acids of L or D configuration, if applies; CCC should contain a trifunctional amino acid capable of forming a covalent bond, preferably ester, thioester, or amide, with the carboxyl function of the C-terminal amino acid of peptide EEE. CCC, DDD, and EEE form part of a cycle, and the process involves extending the exocyclic chain.

In a preferred aspect, the invention involves a synthetic strategy consisting of adding amino acids BBB of the exocylic chain to the cyclic part of the compound where CCC, DDD, and EEE form the cycle. The amino acids can be added one by one, or in fragments that contain two or more amino acids including the terminal aliphatic/fatty acid AAA.

In a preferred synthesis, two fragments are separately constructed for example using solid phase synthesis, one containing at least the cyclic part optionally with one or more of the sidechain amino acids, and one containing at least the terminal acid attached to one or more sidechain amino acids. The fragments can be cleaved from the solid phase and joined.

In particular, we can consider the synthesis of compounds of formula (1): 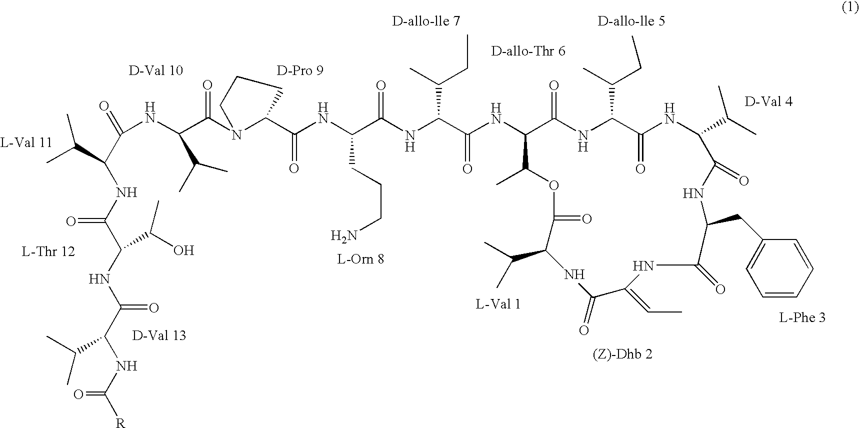
wherein RCO is a terminal acyl and R is preferably a branched alkyl group, especially an alkyl group of 6 to 8 carbon atoms and with a single methyl branch. Examples of RCO are 5-methylhexanoyl or 4(S)-methylhexanoyl.

The most preferred strategy involves making the join between D-Pro-9 and L-Orn-8. There are other candidate strategies that are possible such as L-Orn-8 and D-allo-Ile-7, and also D-Val-10 and D-Pro-9, D-allo-Ile-7 and D-allo-Thr-6 or L-Val- 1 and D-Val-10.

Following the coupling of all the molecule it is also possible to carry out dehydration and deprotection reactions in order to obtain the desired final compound.

Another possibility for generating the fragment including the cyclic part is to start with natural KF, and treat it with trypsin (it will cut by the Orn). It is then possible to perform the coupling with the natural fragment and a non natural fragment (the one with the acyl group).

The preferred embodiment of the synthetic process of the present invention is best represented in the Scheme II, which is directed to the formation of the target compound with a 4(S)-methylhexyl terminal acid. 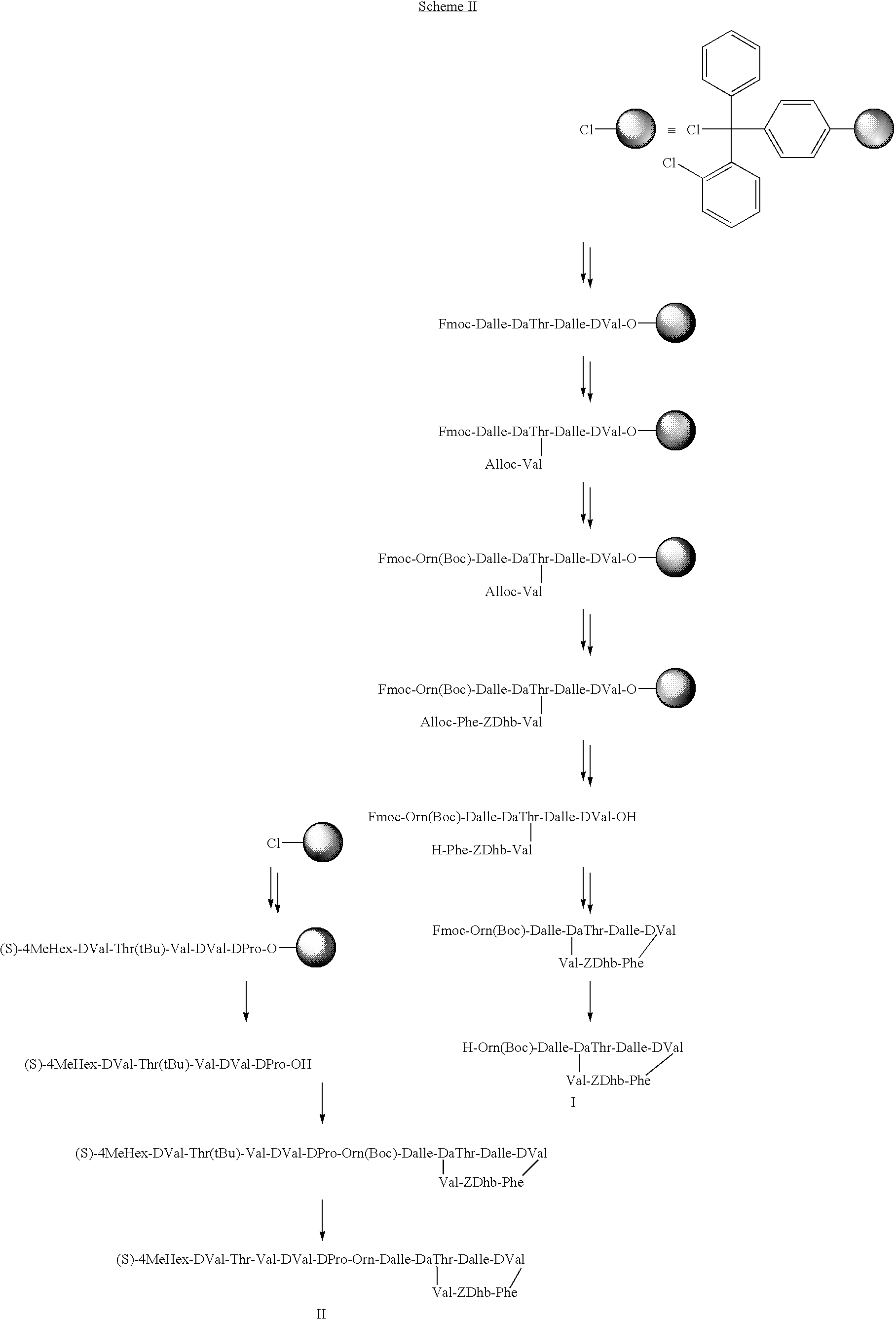
The key steps of the optimized process for a more economical and safe synthesis of kahalalide analogues are: (i) preparation of the two protected peptides onto a chlorotritylchloro-polystyrene resin or related resin; (ii) the C-terminal residue at the N-terminal protected peptide is Pro, which is the least prone to be racemized during the coupling reagent; (iii) for the stepwise synthesis of both protected peptides, use of DIPCDIC-HOBt as coupling method instead of HATU-DIPEA, for the sequential incorporation of the protected amino acids and aliphatic carboxylic acids; (iv) use of sodium diethyl-dithiocarbamate after removing Alloc to avoid presence of Pd (0) in the final product; (v) cyclization step with DIPCDI/HOBt/DIPEA in CH₂Cl₂; these conditions avoids two side reaction: epimerisation of the Val residue, which is involved in the activation, and trifluoroacetylation of the Phe or its replacement; (vi) removal of the Fmoc group in such conditions that leaves inalterated the cyclic; (vii) if needed, purification of both protected peptides; (viii) coupling of both protected peptides with PyAOP/DIEA.

As shown above in Scheme II, the preferred process for the synthetic formation of analogues of Kahalalide F is based in a convergent solid-phase (protected fragment syntheses) and solution (cyclization, fragment condensation, and final deprotection) method using an orthogonal protecting scheme based on a Fmoc/tBu strategy, see for example Lloyd-Williams, P., et al. Chemical Approaches to the Synthesis of Peptides and Proteins. CRC Press, Boca Raton (FL), 1997.

The process of Scheme II comprises the sequential steps of:

Protected Fragment I

(a) incorporating an Fmoc-DVal-OH onto a chlorotrityl chloro resin, forming an ester bond;

(b) elongating the peptidic chain with three amino acids [Dalle, DaThr (free OH), DaIle) using a Fmoc/tBu strategy;

(c) incorporating [Val(l)] using an Alloc as protecting group;

(d) incorporating Fmoc-Orn(Boc)-OH;

(e) incorporating the dipeptide Alloc-Phe-ZDhb-OH, which has been combined and dehydrated in solution;

(f) removing the Alloc, or of its replacement, while the peptide is still anchored to the solid support;

(g) cleaving the side-chain protected peptide from the solid support;

(h) cyclizing the peptide in solution;

(i) removing the Fmoc group of the Orn.

Alternatively, protected fragment can be prepared starting by

(a′) incorporating Alloc-Phe-ZDhb-OH, which has been combined and dehydrated in solution, onto a chlorotrityl chloro resin, forming an ester bond;

(b′) elongating the peptidic chain with five amino acids [Fmoc-DVal-OH, Frmoc-DaIle-OH, Fmoc-DaThr-OH (free OH), Fmoc-DaIle-OH, and Alloc-Orn(Boc)-OH];

(c′) incorporating Val(1) using a Fmoc as protecting group;

(d′) removing the Fmoc while the peptide is still anchored to the solid support;

(e′) cleaving the side-chain protected peptide from the solid support;

(g′) cyclizing the peptide in solution;

(h′) removing the Alloc group of the Orn.

This alternate strategy shows the following advantages:

(i) as the incorporation onto a chlorotrityl chloro resin requires a defect of protected building block, the amount of the precious Alloc-Phe-ZDhb-OH used is less if compared with the first strategy,

(ii) possibility of preparing Fmoc-Phe-Z-Dhb-OH (avoiding a Pd treatment),

(iii) minimization or removal of racemization during the cyclization, and

(iv) stability of cycle to Pd(0) used to remove the Alloc (step h′).

Protected Fragment II

(a) incorporating an Fmoc-DPro-OH onto a chlorotrityl chloro resin, forming an ester bond;

(b) elongating the peptidic chain using a Fmoc/ tBu strategy;

(c) cleaving the side-chain protected peptide from the solid support;

Final Steps

(i) fragment condensation

(ii) final deprotection

Therefore the process can be conducted as follows:

Fmoc-DVal-OH or Alloc-Phe-ZDhb-OH, which was prepared in solution from Alloc-Phe-OH and H-Thr-OtBu with EDC,HCl, and posterior dehydration and treatment with TFA, are incorporated preferably to a chlorotrityl-polystyrene resin, see Barlos, K.; Gatos, D.; Schäfer, W. Angew. Chem. Int. Ed. Engl. 1991, 30, 590-593.

Removal of the Fmoc group can be carried out with piperidine-DMF (2:8, v/v) (1×2 min, 2×10 min). Couplings of Fmoc-aa-OH (4-5 equiv) (aa means amino acid) can be carried out with DIPCDI-HOBt (equimolar amounts of each one respect to the carboxylic component) or PyBOP-DIPEA (equimolar amount of PyBOP and double amount of DIPEA) in DMF or DMF-Toluene (1:1) for 90 min. After the coupling ninhydrin or chloranil tests are carried out and if it is positive the coupling is repeated in the same conditions, otherwise the process is continued. Washings between deprotection, coupling, and, again, deprotection steps can be carried out with DMF (5×0.5 min) and CH₂Cl₂ (5×0.5 min) using for example each time 10 mL solvent/g resin.

Incorporation of Alloc-Val-OH (5 equiv) can be carried out with equimolar amount of DIPCDI and 10% of DMAP. This coupling is repeated at least twice.

Removal of Alloc group can be carried out with Pd(PPh₃)₄ (0.1 equiv) in the presence of PhSiH₃ (10 equiv), see Gómez-Martinez P., Thieriet N., Albericio F., Guibé F. J. Chem. Soc. Perkin I 1999, 2871-2874, and washing the resin with sodium diethyldithiocarbamate in DMF 0.02 M (3×15 min).

In the first strategy, the dipeptide Alloc-Phe-ZDhbOH (4 equiv), can be coupled to the Val (1) residue anchored to the resin with DIPCDI-HOAt (4 equiv of each) for 5 h to overnight.

Cleavage of the protected peptide from the resin can be accomplished by TFA-CH₂Cl₂ (1:99) (5×30 sec).

Cyclization step can be carried out with DIPCDI/HOBt/DIPEA in CH₂Cl₂. These conditions avoid two side reactions: epimerisation of the Val residue, which is involved in the activation, and trifluoroacetylation of the Phe or its replacement.

In the first strategy, removal of the Fmoc group of the Orn residue with 20 equiv of dimethylamine minimize the opening of the cyclic.

Fragment condensation is carried out with PyAOP/DIPEA (equimolar amount of PYAOP with respect to carboxylic component and 3 equivalents of DIPEA). After 1 hour, is used another equimolar amount of PyAOP is used (until the condensation is completed).

Final deprotection can be carried out with TFA-H₂O (95:5) for 1 h.

It will be appreciated that the particular choice of protecting groups is not critical, and other choices are widely available. For example, Bzl type groups can replace tBu/Boc; Boc instead of Fmoc; pNZ instead of Fmoc of Orn; Fmoc instead of Alloc; Alloc instead of Fmoc; Wang resin instead of chlorotrityl. Those protecting groups and resins are described in Greene and Wuts, Protective Groups in Organic Synthesis, John Wiley & Sons, Inc, New York, 1999 and Goodman, M. ed. Houben-Weyl, Methods of Organic Chemistry, Vol. E 22A, Synthesis of Peptides and Peptidomimetics, Thieme, Stuggart-New York, 2001.

Further detail on the synthesis is given in the examples.

The process of this invention can be carried out from starting materials in an enantio-, stereocontrolled and fast manner, taking advantages of the solid-phase synthetic methodology, where the molecule in construction is bounded to an insoluble support during all synthetic operations.

### EXAMPLES

General Procedures. Cl-TrtCl-resin, Protected Fmoc-amino acid derivatives, HOBt, HOAt were from ABI (Framingham, Mass.), Bachem (Bubendorf, Switzerland), NovaBiochem (Läufelfingen, Switzerland), and IRIS Biotech (Marktredwitz, Germany). 4(S)-MeHex derivatives from Narchem.

Alloc-amino acids were prepared essentially as described by Dangles et al. (see J. Org. Chem. 1987, 52, 4984-4993) and Cruz et al. (see Org. Proc. Res. Develop. 2004, 8, 920-924). Alloc-ZDhb-Phe-OH was prepared as described in WO 01 58934, and DIPEA, DIPCDI, EDC-HCl, Piperidine, TFA were from Aldrich (Milwaukee, Wis.). DMF and CH₂Cl₂ were from SDS (Peypin, France). ACN (HPLC grade) was from Scharlau (Barcelona, Spain). All commercial reagents and solvents were used as received with exception of CH₂Cl₂, which was passed through a alumina column to remove acidic contaminants.

Solid-phase syntheses were carried out in polypropylene syringes (10-50 mL) fitted with a polyethylene porous disc. Solvents and soluble reagents were removed by suction. Removal of the Fmoc group was carried out with piperidine-DMF (2:8, v/v) (1×2 min, 2×10 min). Washings between deprotection, coupling, and, again, deprotection steps were carried out with DMF (5×0.5 min) and CH₂Cl₂ (5×0.5 min) using each time 10 mL solvent/g resin. Peptide synthesis transformations and washes were performed at 25° C. Synthesis carried out on solid-phase were controlled by HPLC of the intermediates obtained after cleaving with TFA-H20 (1:99) for 1 min an aliquot (aprox. 2 mg) of the peptidyl-resin. HPLC reversed phase columns Symmetry™ C₁₈ 4,6×150 mm, 5 μm (column A) and Symmetry300™ C₁₈ 4,6×50 mm, 5 μm (column B) were from Waters (Ireland). Analytical HPLC was carried out on a Waters instrument comprising two solvent delivery pumps (Waters 1525), automatic injector (Waters 717 autosampler), dual wavelength detector (Waters 2487), and system controller (Breeze V3.20) and on a Agilent 1100 instrument comprising two solvent delivery pumps (G1311A), automatic injector (G1329A), DAD (G1315B). UV detection was at 215 or 220 nm, and linear gradients of CH₃CN (+0.036% TFA) into H₂O (+0.045% TFA), from 30% to 100% in 15 min. MALDI-TOF and ES-MS analysis of peptide samples were performed in a PerSeptive Biosystems Voyager DE RP, using ACH matrix, and in a Waters Micromass ZQ spectrometer and in an Agilent Ion Trap 1100 Series LC/MSDTrap. Peptide-resin samples were hydrolyzed in 12 N aqueous HCl-propionic acid (1:1), at 155° C. for 1-3 h and peptide-free samples were hydrolyzed in 6 N aqueous HCl at 155° C. for 1 h. Subsequent amino acid analysis was performed on a Beckman System 6300 autoanalyzer.

Nomenclature used for cyclic peptides and precursors as described by Spengler et al. (see Spengler J., Jimenez J. C., Burger K., Giralt E., Albericio, F. “Abbreviated nomenclature for cyclic and branched homo- and hetero-detic peptides”. J. Peptide Res. 2005, on line publication: 13-Apr-2005). The “&” symbol is used in the nomenclature for cyclic peptides and precursors. The appearance of “&,” in a given position of the one-line formula represents the point at which one end of a chemical bond is located and the second “&” indicates the point to which this bond is attached. Thus, “&” represents the start or the end of a chemical bond, which is ‘cut’ with the aim of visualizing a complex formula more easily. In this way, two “&” symbols represent one chemical bond.

### Example 1

### [H-Orn(Boc)-D-allo-Ile-D-allo-Thr(&)-D-allo-Ile-D-Val-OH][H-Phe-(Z)Dhb-Val&] from Fmoc-Orn(Boc)-OH

### Step 1

H-D-Val-O-TrtCl-resin.

Cl-TrtCl-resin (1 g, 1.64 mmol/g) was placed in a 20 mL polypropylene syringe fitted with a polyethylene filter disk. The resin was then washed with CH₂Cl₂ (5×0.5 min), and a solution of Fmoc-D-Val-OH (238 mg, 0.7 mmol, 0.43 equiv) and DIPEA (0.41 mL) in CH₂Cl₂ (2.5 mL) was added, and the mixture was stirred for 15 min. Then extra DIPEA (0.81 mL, total 7 mmol) was added and the mixture was stirred during 45 min more. The reaction was terminated by addition of MeOH (800 μL), after a stirring of 10 min. The Fmoc-D-Val-O-TrtCl-resin was subjected to the following washings/treatments with CH₂Cl₂ (3×0.5 min), DMF (3×0.5 min), piperidine as indicated in General Procedures, and DMF (5×0.5 min). The loading calculated by Fmoc determination was 0.50 mmol/g.

### Step 2

[Fmoc-D-allo-Ile-D-allo-Thr(&)-D-allo-Ile-D-Val-O-TrtCl-resin][Alloc-Val&].

Fmoc-D-allo-Ile-OH (707 mg, 2 mmol, 4 equiv), Fmoc-D-allo-Thr-OH (free hydroxy group) (683 mg, 2 mmol, 4 equiv), and Fmoc-D-allo-Ile-OH (707 mg, 2 mmol, 4 equiv) were added sequentially to the above obtained H-D-Val-O-TrtCl-resin using DIPCDI (310 μL, 2 mmol, 4 equiv) and HOBt (307 mg, 2 mmol, 4 equiv) in DMF (2.5 mL). In all cases, after 90 min of coupling, the ninhydrin test was negative. Removal of Fmoc group and washings were carried out as described in General Procedures. Alloc-Val-OH (502 mg, 2.5 mmol, 5 equiv) was coupled with DIPCDI (387 mg, 2.5 mmol, 5 equiv) in the presence of DMAP (30.6 mg, 0.25 mmol, 0.5 equiv) and DIPEA (88 μL, 0.5 mmol, 1 equiv) for 45 min. This coupling was repeated in the same conditions twice. An aliqout of the peptidyl-resin was treated with TFA and the HPLC (t_{R} 14.2 min, column A) of the crude obtained after evaporation showed a purity of >98%.

ESMS, calcd for C₄₅H₆₃N₅O1₁, 849.45. Found: m/z 850.1 [M+H]⁺, 871.9 [M+Na]⁺.

### Step 3

[Fmoc-Orn(Boc)-D-allo-Ile-D-allo-Thr(&)-D-allo-Ile-D-Val-O-TrtCl-resin][Alloc-Val&,].

The Fmoc group of the above peptidyl-resin (Step 2) was removed and Fmoc-Orn(Boc)-OH (912 mg, 2 mmol, 4 equiv) was added using DIPCDI (310 μL, for 2.0 mmol and 4 equiv; and 388 μL, for 2.5 mmol and 5 equiv) and HOBt (307 mg, for 2.0 mmol and 4 equiv; and 395 mg, for 2.5 mmol and 5 equiv) for 90 min. Ninhydrin test after the incorporation was negative. An aliqout of the peptidyl-resin was treated with TFA and the HPLC (t_{R} 12.8 min, column A) of the crude obtained after evaporation showed a purity of 90%.

ESMS, calcd for C₅₆H₈₁N₇O₁₄, 1,063.58. Found: m/z 1,086.77 [M+Na⁺]⁺.

### Step 4

[Fmoc-Orn(Boc)-D-allo-Ile-D-allo-Thr(&)-D-allo-Ile-D-Val-O-TrtCl-resin][Alloc-Phe-ZDhb-Val&,].

Alloc group of the above peptidyl-resin (Step 3) was removed with Pd(PPh₃)₄ (58 mg, 0.05 mmol, 0.1 equiv) in the presence of PhSiH₃ (617 μL, 5 mmol, 10 equiv), followed by washings with diethyldithiocarbamate 0.02 M (3×15 min). Alloc-Phe-ZDhb-OH (666 mg, 2 mmol, 4 equiv) and HOAt (273 mg, 2 mmol, 4 equiv) were dissolved in DMF (1.25 mL) and added to peptidyl-resin. Then DIPCDI (310 μL, 2 mmol, 4 equiv) was added and the mixture stirred for 5 h, where the ninhydrin test was negative. After washings with DMF and CH₂Cl₂, an aliquot of the peptidyl-resin was treated with TFA-H₂O (1:99) for 1 min and the product was characterized by MALDI-TOF-MS.

MALDI-TOF-MS, calcd for C₆₈H₉₅N₉O₁₆, 1,293.69 Found: m/z 1,294.35 [M+H]⁺, 1,316.39 [M+Na]⁺, 1,333.34 [M+K]⁺.

### Step 5

[Fmoc-Orn(Boc)-D-allo-Ile-D-allo-Thr(&)-D-allo-Ile-D-Val-OH][H-Phe-ZDhb-Val&].

Alloc group of the above peptidyl-resin (Step 4) was removed with Pd(PPh₃)₄ (58 mg, 0.05 mmol, 0.1 equiv) in the presence of PhSiH₃ (617 μL, 5 mmol, 10 equiv), the resin was washed with sodium diethyldithiocarbamate in DMF 0.02 M (3×15 min) and the protected peptide was cleaved from the resin by TFA-CH₂Cl₂ (1:99) (5×30 sec). Filtrate was collected on H₂O (4 mL) and the H₂O was partially removed under reduced pressure. ACN was then added to dissolve solid that appeared during the H₂O removal, and the solution was lyophilized, to give 700 mg (578 μmol, 99% yield of the title compound with a purity of >91% as checked by HPLC (Column A, t_{R} 8.59 min).

MALDI-TOF-MS, calcd for C₆₄H₉₁N₉O₁₄, 1,209.67. Found: m/z 1,210.45 [M+H]⁺, 1,232.51 [M+Na]⁺, 1,248.45 [M+K]⁺.

### Step 6

Fmoc-Orn(Boc)-D-allo-Ile-D-allo-Thr(&)-D-allo-Ile-D-Val-Phe-ZDhb-Val&.

The protected peptide (Step 5) (700 mg, 578 μmol) was dissolved in CH₂Cl₂ (580 mL, 1 mM), and HOBt (137 mg, 2.3 mmol) dissolved in the minimum volume of DMF to dissolve HOBt, DIPEA (302 μL, 1.73 mmol, 3 equiv), and DIPCDI (356 μL, 2.3 mmol, 4 equiv) were added. The mixture was allowed to stir for 1 h, and then the course of the cyclization step was checked by HPLC (column A, t_{R} 12.4 min). The solvent was removed by evaporation under reduced pressure.

MALDI-TOF-MS, calcd for C₆₄H₈₉N₉O₁₃, 1,191.66. Found: m/z 1,092.17 [M+H]⁺, 1,214.14 [M+Na]⁺, 1,230.10 [M+K]⁺.

### Step 7

H-Orn(Boc)-D-allo-Ile-D-allo-Thr(&)-D-allo-Ile-D-Val-Phe-ZDhb-Val&

The protected peptide (Step 6) (50 mg, 42 μmol) was dissolved in DMF (5 mL), then diethylamine (130 μL, 30 equiv) was added and the mixture was left to stir by 1:30 min. The solvent was removed by evaporation under reduced pressure. The crude product was purified by HPLC (Symmetry C₈ 5 [μm, 30×100 mm), linear gradient of ACN (30% to 75% in 15 min) ACN (+0.05% TFA) in water (+0.05% TFA), 20 mL/h, detection at 220 nm. The product was characterized by HPLC (t_{R} 8.7 min, Condition A) and for MALDI-TOF-MS, calcd C₄₉H₇₉N₉O₁₁, 969.59. Found: m/z 970.87 [M+H]⁺, 870.78 [M-Boc]⁺.

### Example 2

### [H-Orn(Boc)-D-allo-Ile-D-allo-Thr(&)-D-allo-Ile-D-Val-OH][H-Phe-(Z)Dhb-Val&] from NZ-Orn(Boc)-OH

Experimental procedures as described in Examples 1, except that in the step 3, Fmoc-Orn-OH is replaced by pNZ-Orn-OH.

The protected peptide (14.7 mg, 12.8 μmol) was dissolved in 1.6 mM HCl in DMF (10 mL), then SnCl₂ (3.8 g, 20 mmol) was added and the mixture was left to stir until HPLC (Column A) showed the completion of the reaction (1 h). The solvent was removed by evaporation under reduced pressure. The crude product was purified by HPLC (Symmetry C₈ 5 μm, 30×100 mm), gradient of ACN (30% to 75% in 15 min) ACN (+0.05% TFA) in water (+0.05% TFA), 20 mL/h, detection at 220 nm, to give the title product (4.8 mg, 4.9 pmol, 40% yield. The product was characterized by HPLC (t_{R} 8.2 min, Column A) and for MALDI-TOF-MS.

MALDI-TOF-MS, calcd C₄₉H₇₉N₉O₁₁, 969.59. Found: m/z 992.35 [M+Na]⁺, 870.34 [M-Boc]⁺, 892.34 [M+Na-Boc]⁺.

### Example 3

### [H-Orn(Boc)-D-allo-Ile-D-allo-Thr(&)-D-allo-Ile-D-Val-OH][H-Phe-(Z)Dhb-Val&] from Alloc-Orn(Boc)-OH

### Step 1

H-Phe-(Z)Dhb-O-TrtCl-resin.

Cl-TrtCl-resin (1 g, 1.64 mmol/g) was placed in a 20 mL polypropylene syringe fitted with a polyethylene filter disk. The resin was then washed with CH₂Cl₂ (5×0.5 min), and a solution of Alloc-Phe-(Z)Dhb-OH (232 mg, 0.7 mmol, 0.42 equiv) and DIPEA (0.41 mL) in CH₂Cl₂ (2.5 mL) was added, and the mixture was stirred for 15 min. Then extra DIPEA (0.81 mL, total 7 mmol) was added and the mixture was stirred during 45 min more. The reaction was terminated by addition of MeOH (800 μL), after a stirring of 10 min. The Alloc-Phe-(z)Dhb-O-TrtCl-resin was subjected to the following washings with CH₂Cl₂ (3×0.5 min), DMF (3×0.5 min), and the Alloc group was removed with Pd(PPh₃)₄ (58 mg, 0.05 mmol, 0.1 equiv) in the presence of PhSiH₃ (617 μL, 5 mmol, 10 equiv) in CH₂Cl₂. The resin was washed as described in General Procedures. The loading calculated by Fmoc determination was 0.68 mmol/g.

### Step 2

[Alloc-Orn(Boc)-D-allo-Ile-D-allo-Thr(&)-D-allo-Ile-D-Val-Phe-(Z)Dhb-OH][H-Val&]

Fmoc-D-Val-OH (678 mg, 2 mmol, 4 equiv), Fmoc-D-allo-Ile-OH (707 mg, 2 mmol, 4 equiv), Fmoc-D-allo-Thr-OH (free hydroxy group) (683 mg, 2 mmol, 4 equiv), Fmoc-D-allo-Ile-OH (707 mg, 2 mmol, 4 equiv), and Alloc-Orn(Boc)-OH (630 mg, 2 mmol, 4 equiv) were added sequentially to the above obtained H-Phe-(Z)Dhb-O-TrtCl-resin using DIPCDI (310 μL, 2 mmol, 4 equiv) and HOBt (307 mg, 2 mmol, 4 equiv) in DMF (2.5 mL). In all cases, after 90 min of coupling, the ninhydrin test was negative. Removal of Fmoc group and washings were carried out as described in General Procedures. Fmoc-Val-OH (848.2 mg, 2.5 mmol, 5 equiv) was coupled with DIPCDI (387 mg, 2.5 mmol, 5 equiv) in the presence of DMAP (30.6 mg, 0.25 mmol, 0.5 equiv) and DIPEA (88 μL, 0.5 mmol, 1 equiv) for 45 min. This coupling was repeated in the same conditions twice. After removal of the Fmoc group as described in General Procedures, the protected peptide was cleaved from the resin by TFA-CH₂Cl₂ (1:99) (5×30 sec). Filtrate was collected on H₂O (4 mL) and the H₂O was partially removed under reduced pressure. ACN was then added to dissolve the solid that appeared during the H₂O removal, and the solution was lyophilized, to give 650 mg (606 μmol, 90% yield of the title compound with a purity of >75% as checked by HPLC (Column A, t_{R} 9.93 min).

ESMS, calcd for C₅₃H₈₅N₉O₁₄, 1072, 29. Found: m/z 1074,4 [M+H]⁺.

### Step 3

Alloc-Orn(Boc)-D-allo-Ile-D-allo-Thr(&)-D-allo-Ile-D-Val-Phe-ZDhb-Val&.

The protected peptide (Step 2) (250 mg, 0.233 mmol) was dissolved in CH₂Cl₂ (240 mL, 1 mM), and HOAt (126 mg, 9.325 mmol, 4 equiv) dissolved in the minimum volume of DMF to dissolve HOAt, and DIPCDI (143 μL, 9,325 mmol, 4 equiv) were added. The mixture was allowed to stir for 24 h, then the course of the cyclization step was checked by HPLC (column A, t_{R} 12.82 min). The solvent was removed by evaporation under reduced pressure.

MALDI-TOF-MS, calcd for C₅₃H₈₃N₉O₁₃, 1,054.28. Found: m/z 1056.4 [M+H]⁺.

### Step 4

H-Orn(Boc)-D-allo-Ile-D-allo-Thr(&)-D-allo-Ile-D-Val-Phe-ZDhb-Val&.

The protected peptide (Step 3) (244 mg, 231 μmol) was dissolved in 10 mL of CH₂Cl₂, then Pd(PPh₃)₄ (8 mg, 6.94 μmol, 0.03 equiv) in the presence of PhSiH₃ (94 μL, 763.6 μmol, 3.3 equiv) was added and the mixture was left to stir by 1:30 min. The solvent was removed by evaporation under reduced pressure. The crude product was purified by HPLC (Symmetry C₈ 5 Mm, 30×100 mm), linear gradient of ACN (20% to 80% in 15 min) ACN (+0.05% TFA) in water (+0.05% TFA), 20 mL/h, detection at 220 nm. The product was characterized by HPLC (t_{R} 9.19 min, Condition A) and for MALDI-TOF-MS, calcd C₄₉H₇₉N₉O₁₁, 970.21. Found: m/z 972.1 [M+H]⁺.

### Example 4

### (4S)-MeHex-D-Val-Thr(tBu)-Val-D-Val-D-Pro-OH

### Step 1

H-D-Pro-O-TrtCl-resin.

Cl-TrtCl-resin (1 g, 1.64 mmol/g) was placed in a 20 mL polypropylene syringe fitted with a polyethylene filter disk. The resin was then washed with CH₂Cl₂ (5×0.5 min), and a solution of Fmoc-D-Pro-OH (237 mg, 0.7 mmol, 0.43 equiv) and DIPEA (0.41 mL) in CH₂Cl₂ (2.5 mL) was added, and the mixture was stirred for 15 min. Extra DIPEA (0.81 mL, total 7 mmol) was added and the mixture was stirred for 45 min. The reaction was terminated by addition of MeOH (800 μL), after a stirring of 10 min. The Fmoc-D-Pro-O-TrtCl-resin was subjected to the following washings/treatments with CH₂Cl₂ (3×0.5 min), DMF (3×0.5 min), piperidine as indicated in General Procedures, and DMF (5×0.5 min). The loading calculated by Fmoc determination was 0.27 mmol/g.

### Step 2

(4S)-MeHex-D-Val-Thr(tBu)-Val-D-Val-D-Pro-OH

Fmoc-D-Val-OH (458 mg, 1.32 mmol, 5 equiv), Fmoc-Val-OH (360 mg, 1.06 mmol, 4 equiv), Fmoc-Thr(tBu)-OH (527 mg, 1.32 mmol, 5 equiv), Fmoc-D-Val-OH (360 mg, 1.06 mmol, 4 equiv), and (4S-MeHex-OH (138 mg, 1.06 mmol, 4 equiv) were sequentially added to the above peptidyl-resin (Step 1) using DIPCDI (165 μL, for 1.06 mmol and 4 equiv; and 205 μL, for 1.32 mmol and 5 equiv) and HOBt (162 mg, for 1.06 mmol and 4 equiv; and 203 mg, for 1.32 mmol and 5 equiv) for 90 min. In all cases, after 90 min of coupling, the ninhydrin test was negative. Removal of Fmoc group and washings were carried out as described in General Procedures.

The partial protected peptide was cleaved from the resin by TFA-CH₂Cl₂ (1:99) (5×30 sec). Filtrate was collected on H₂O (4 mL) and the H₂O was partially removed in a rotavapor. ACN was then added to dissolve the solid that appeared during the H₂O removal, and the solution was lyophilized, to give 154.4 mg (226 μmol, 85.5% yield) of the title compound with a purity of >94% as checked by HPLC (Column A, t_{R} 12.13 min). The crude obtained after evaporation showed a purity of >94%. The product was characterized by Electrospray.

Calcd for C₃₅H₆₃N₅O₈, 681.9. Found: m/z 682.15.

### Example 5

### (4S)-MeHex-D-Val-Thr(tBu)-Val-D-Val-D-Pro-Orn(Boc)-D-allo-Ile-D-allo-Thr(&)-D-allo-Ile-D-Val-Phe-ZDhb-Val&

Peptides from Examples 1 (8.25 mg, 8.5 μmol) and 4 (7 mg, 10.2 μmol, 1.2 equiv) were dissolved in DMF (10 mL) and PyAOP (5.32 mg, 10.2 μmol, 1.2 equiv) and DIPEA (5.3 μl, 30.6 μmol, 3.6 equiv) were added at room temperature. The mixture was stirred for 1 h, when extra PyAOP (5.32 mg, 10.2 μmol, 1.2 equiv) were added. The mixture was allowed to react for 2 h at room temperature, until HPLC (Column A) showed the completion of the reaction. The crude obtained after evaporation showed by HPLC a purity of >75%. The crude product was purified by HPLC (Symmetry C₈ 5 μm, 30×100 mm), linear gradient of ACN (+0.05% TFA) in water (+0.05% TFA) (30% to 100% in 15 min), 20 mL/h, detection at 220, to give the title product (6.9 mg, 4.2 μmol, 49% yield).

MALDI-TOF-MS, calcd for C₈₄H₁₄₀N₁₄O₁₈, 1.633.05. Found: m/z 1,534.33 [M-Boc]⁺,1,556.26 [M-Boc+Na]⁺ 1,656.33 [M+Na]⁺.

### Example 6

### (4S)-MeHex-D-Val-Thr-Val-D-Val-D-Pro-Orn-D-allo-Ile-D-allo-Thr(&)-D-allo-Ile-D-Val-Phe-ZDhb-Val&

The protected cyclic peptide (Example 5) was dissolved in TFA-H₂O (19:1, 700 μL) and the mixture was allowed to stir for 1 h. The solvent was removed by evaporation under reduced pressure, and dioxane was added (245 μL). The solvent was removed by evaporation under reduced pressure (the process was repeated three times), and then H₂O (1 mL) was added and lyophilized. The crude product was purified by HPLC (Symmetry C₈ 5 μm, 30×100 mm), isocratic 44% ACN (+0.05% TFA) in water (+0.05% TFA), 20 mL/h, detection at 220 nm, to give the title product (5 mg, 3.4 μmol, 80% yield, 93.3%). The HPLC did not show the presence of the epimeric peptide (4S)-MeHex-D-Val-Thr-Val-D-Val-Pro-Orn-D-allo-Ile-D-allo-Thr(&)-D-allo-Ile-D-Val-Phe-ZDhb-Val& (Example 7), which would indicate racemization during the coupling step between both protected peptides.

MALDI-TOF-MS, calcd for C₇₅H₁₂₄N₁₄O₁₆, 1,476.93. Found: m/z 1,478.17 [M+H]⁺ 1,500.14 [M+Na]⁺, 1,516.12 [M+K]⁺.

### Example 7

### (4S)-MeHex-D-Val-Thr-Val-D-Val-Pro-Orn-D-allo-Ile-D-allo-Thr(&)-D-allo-Ile-D-Val-Phe-ZDhb-Val&

Experimental procedures as described in Examples 1,4,5 and 6, except that in the step 1 of Example 4 Fmoc-D-Pro-OH is replaced by Fmoc-Pro-OH. The product was characterized by HPLC (t_{R} 11.23 min, Column A) and for MALDI-TOF-MS.

MALDI-TOF-MS, calcd C₇₅H₁₂₄N₁₄O₁₆, 1,476.93. Found: m/z 1,500.23 [M+Na]⁺, 1,515.97 [M+K]⁺.

### Example 8

### H-D-allo-Ile-D-allo-Thr(&)-D-allo-Ile-D-Val-Phe-(Z)Dhb-Val&

Starting with [Fmoc-D-allo-Ile-D-allo-Thr(&)-D-allo-Ile-D-Val-O-TrtCl-resin][Alloc-Val&] (Step 2, Example 1), the Alloc group was removed with Pd(PPh₃)₄ (58 mg, 0.05 mmol, 0.1 equiv) in the presence of PhSiH₃ (617 μL, 5 mmol, 10 equiv) and the resin washed with sodium diethyldithiocarbamate in DMF 0.02 M (3×15 min). Alloc-Phe-Z-Dhb-OH (666 mg, 2 mmol, 4 equiv) and HOAt (273 mg, 2 mmol, 4 equiv) were dissolved in DMF (1.25 mL) and added to peptidyl-resin. Then DIPCDI (310 μL, 2 mmol, 4 equiv) was added and the mixture stirred for 5 h, when the HPLC showed the completion of reaction (t_{R} 7.09 min, Column A).

The Fmoc group was removed and after extensive DMF washings, Boc₂O (546 mg, 5 equiv) and DIPEA (0.87 mL, 10 equiv) in DMF were added and left to react for 2 h, when the ninhydrin test was negative. After DMF washings, the Alloc group was removed as above and the protected peptide was cleaved from the resin with TFA-CH₂Cl₂ (1:99) (5×30 sec). Filtrate was collected on H₂O (4 mL) and the H₂O was partially removed under reduced pressure. ACN was then added to dissolve solid that appeared during the H₂O removal, and the solution was lyophilized.

Cyclization was carried out as in Step 6 of Example 1; and then the Boc was removed with TFA-H₂O (19:1) (1 h). The solvent was removed under reduced pressure and dioxane was added (245 μL). The solvent was removed by evaporation under reduced pressure (the process was repeated three times), and then H₂O (1 mL) was added and lyophilized. The crude product was purified by HPLC (Symmetry C₈ 5 μm, 30×100 mm), isocratic 44% ACN (+0.05% TFA) in water (+0.05% TFA), 20 mL/h, detection at 220 nm, to give the title product (207 mg, 273 μmol, 55% yield, 93.3%).

The product was characterized by HPLC (t_{R} 7.27 min, Column A) and for MALDI-TOF-MS.

MALDI-TOF-MS, calcd C₃₉H₆₁N₇O₈, 755.46. Found: m/z 756.56 [M+H]⁺, 778.55 [M+Na]⁺, 794.53 [M+K]⁺.

### Example 9

### (4S)-MeHex-D-Val-Thr(tBu)-Val-D-Val-D-Pro-Orn(Boc)-OH

Experimental procedures as described in Example 4, except that the peptide synthesis was initiated by incorporation of Fmoc-Orn(Boc)-OH to the Cl-TrtCl-resin. The product was characterized by HPLC (t_{R} 13.27 min, Column A) and for Electrospray.

Calcd C₃₆H₆₅N₇O₉, 739.48. Found: m/z 740.65.

### Example 10

### (4S)-MeHex-D-Val-Thr-Val-D-Val-D-Pro-Orn-D-allo-lie-D-allo-Thr(&)-D-allo-Ile-D-Val-Phe-ZDhb-Val&

Experimental procedures as described in Examples 5 and 6, except that in Example 5 peptide from Example 1 is replaced by peptide from Example 8 and peptide from Example 4 is replaced by peptide from Example 9. The HPLC of the final product showed the presence of the epimeric peptide (4S)-MeHex-D-Val-Thr-Val-D-Val-D-Pro-D-Orn-D-allo-Ile-D-allo-Thr(&)-D-allo-Ile-D-Val-Phe-ZDhb-Val& (4.1%) (Example 11), which indicates racemization during the coupling step between both protected peptides. The product was characterized by HPLC (t_{R} 10.5 min, Column A).

MALDI-TOF-MS, calcd C₇₅H₁₂₄N₁₄O₁₆, 1,476.93. Found: m/z 1,477.99 [M+H]⁺ 1,499.97 [M+Na]⁺, 1,515.93 [M+K]⁺.

### Example 11

### (4S)-MeHex-D-Val-Thr-Val-D-Val-D-Pro-D-Orn-D-allo-Ile-D-atio-Thr(&)-D-aZlo-Ile-D-Val-Phe-ZDhb-Val&

Experimental procedures as described in Example 10, except that Fmoc-Orn(Boc)-OH is replaced by Fmoc-D-Orn(Boc)-OH. The product was characterized by HPLC (t_{R} 9.89 min, Column A).

MALDI-TOF-MS, calcd C₇₅H₁₂₄N₁₄O₁₆, 1,476.93. Found: m/z 1,478.06 [M+H]⁺ 1,500.15 [M+Na]⁺, 1,516.04 [M+K]⁺.

### Example 12

### H-D-allo-Thr(&)-D-allo-Ile-D-Val-Phe-(Z)Dhb-Val&

Starting with Fmoc-D-allo-Thr-D-allo-Ile-D-Val-Phe-ZDhb-O-TrtCl-resin (Step 2, Example 3), the Fmoc group was removed and after extensive DMF washings, Boc₂O (546 mg, 5 equiv) and DIPEA (0.87 mL, 10 equiv) in DMF were added and left to react for 2 h, when the ninhydrin test was negative. After DMF washings, Fmoc-Val-OH (848.2 mg, 2.5 mmol, 5 equiv) was coupled with DIPCDI (387 mg, 2.5 mmol, 5 equiv) in the presence of DMAP (30.6 mg, 0.25 mmol, 0.5 equiv) and DIPEA (88 μL, 0.5 mmol, 1 equiv) for 45 min. This coupling was repeated in the same conditions twice. After removal of the Fmoc group as described in General Procedures, the protected peptide was cleaved from the resin by TFA-CH₂Cl₂ (1:99) (5×30 sec). Filtrate was collected on H₂O (4 mL) and the H₂O was partially removed under reduced pressure. ACN was then added to dissolve the solid that appeared during the H₂O removal, and the solution was lyophilized, to give 57 mg (75 μmol, 90% yield) of the title compound with a purity of >95% as checked by HPLC (Column A, t_{R} 7.95 min).

ESMS, calcd for C₃₈H₆₀N₆O₁₀, 760.44. Found: m/z 762.3 [M+H]⁺.

Cyclization was carried out as in Step 6 of Example 1; and then the Boc was removed with TFA-H₂O (19:1) (1 h). The solvent was removed under reduced pressure and dioxane was added (245 μL). The solvent was removed by evaporation under reduced pressure (the process was repeated three times), and then H₂O (1 mL) was added and lyophilized. The crude product was purified by HPLC (Symmetry C₈ 5 μm, 30×100 mm), isocratic 30% ACN (+0.05% TFA) in water (+0.05% TFA), 20 mL/h, detection at 220 nm, to give the title product (50.2 mg, 67.6 μmol, 90% yield).

The product was characterized by HPLC (t_{R} 10.61 min, Column A) and for MALDI-TOF-MS, calcd C₃₈H₅₈N₆O₉, 742.43. Found: m/z 743.40 [M+H]⁺, 765.43 [M+Na]⁺, 781.43 [M+K]⁺.

### Example 13

### (4S)-MeHex-D-Val-Thr(tBu)-Val-D-Val-D-Pro-Orn(BOC)-D-allo-Ile-OH

Experimental procedures as described in Example 4, except that the peptide synthesis was initiated by incorporation of Fmoc-D-allo-Ile-OH to the Cl-TrtCl-resin. The product was characterized by HPLC (t_{R} 10.25 min, Column A) and for MALDI-TOF-MS, Calcd C₅₁H₉₂N₈O₁₂, 1,008.68. Found: m/z 1,009.8.

### Example 14

### (4S)-MeHex-D-Val-Thr-Val-D-Val-D-Pro-Orn-D-allo-Ile-D-allo-Thr(&)-D-alto-Ile-D-Val-Phe-ZDhb-Val&

Experimental procedures as described in Examples 5 and 6, except that in Example 5 peptide from Example 1 is replaced by peptide from Example 12 and peptide from Example 4 is replaced by peptide from Example 13. The HPLC of the final product showed the presence of the epimeric peptide (4S)-MeHex-D-Val-Thr-Val-D-Val-D-Pro-Orn-Ile-D-allo-Thr(&)-D-allo-Ile-D-Val-Phe-ZDhb-Val& (Example 15), which indicates racemization during the coupling step between both protected peptides. The product was characterized by HPLC (t_{R} 7.92 min, Column A). MALDI-TOF-MS, calcd C₇₅H₁₂₄N₁₄O₁₆, 1,476.93. Found: m/z 1,478.5 [M+H]⁺ 1,501.4 [M+Na]⁺, 1,517.6 [M+K]⁺.

### Example 15

### (4S)-MeHex-D-Val-Thr-Val-D-Val-D-Pro-Orn-Ile-D-atlo-Thr(&)-D-allo-Ile-D-Val-Phe-ZDhb-Val&

Experimental procedures as described in Examples 12-14, except that Fmoc-Ile-OH was used instead of Fmoc-D-allo-Ile-OH (Example 13).

(4S)-MeHex-D-Val-Thr(tBu)-Val-D-Val-D-Pro-Orn(Boc)-Ile-OH: (t_{R} 10.25 min, Column A and MALDI-TOF-MS, Calcd C₅₁H₉₂N₈O₁₂, 1,008.68. Found: m/z 1,009.5).

The final product was characterized by HPLC (t_{R} 8.02 min, Column A). MALDI-TOF-MS, calcd C₇₅H₁₂₄N₁₄O₁₆, 1,476.93. Found: m/z 1,478.2 [M+H]⁺ 1,501.1 [M+Na]⁺, 1,517.3 [M+K]⁺.

### Example 16

### (4S)-MeHex-D-Val-Thr-Val-D-Val-D-Pro-Orn-D-allo-Ile-D-allo-Thr(&)-D-allo-Ile-D-Val-Phe-ZDhb-D-Val&

Experimental procedures as described in Examples 3,4 and 5 except that Fmoc-D-Val-OH was used instead of Fmoc-Val-OH (Example 3).

Alloc-Orn(Boc)-D-allo-Ile-D-allo-Thr(&)-D-allo-Ile-D-Val-Phe-ZDhb-Val&: t_{R} 12.52 min, column A; MALDI-TOF-MS, calcd for C₅₃H₈₃N₉O₁₃, 1,054.28. Found: m/z 1,056.6 [M+H]⁺.
H-Orn(Boc)-D-allo-Ile-D-allo-Thr(&)-D-allo-Ile-D-Val-Phe-ZDhb-Val&: (t_{R} 9.23 min, Condition A and MALDI-TOF-MS, calcd C₄₉H₇₉N₉O₁₁, 970.21. Found: m/z 972.4[M+H]⁺.

The final product was characterized by HPLC (t_{R} 9.23 min, Column A). MALDI-TOF-MS, calcd C₇₅H₁₂₄N₁₄O₁₆, 1,476.93. Found: m/z 1,479.6 [M+H]⁺ 1,502.5 [M+Na]⁺, 1,518.7 [M+K]⁺.

1. A process for the synthesis of kahalalide F or a mimic of kahalalide F comprising coupling a cyclic part with a side chain fragment.

2. A process according to claim 1, wherein the cyclic part already contains at least one of the side chain amino acids.

3. A process according to claim 1 or 2, wherein the compound obtained is kahalalide F with the following formula:

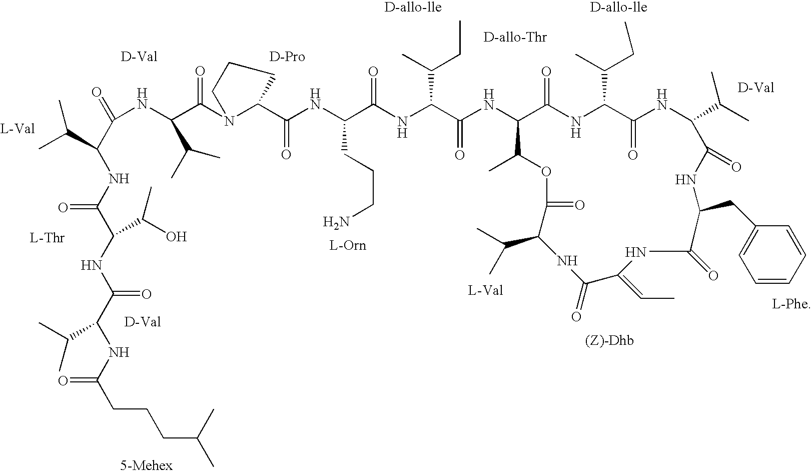

4. A process according to claim 1 or 2, wherein the compound obtained is a mimic of kahalalide F which is structurally related to said compound, but differing in one or more of the following aspects:
1 to 7, especially 1 to 3, more especially 1 or 2, most especially 1, amino acid which is not the same as an amino acid of the parent compound;
1 to 10, especially 1 to 6, more especially 1 to 3, most especially 1 or 2, additional methylene groups in the side chain acyl group of the parent compound;
1 to 10, especially 1 to 6, more especially 1 to 3, most especially 1 or 2, methylene groups omitted from the side chain acyl group of the parent compound;
1 to 6, especially 1 to 3, more especially 1 or 3, substituents added to or omitted from the side chain acyl group of the parent compound;
omission of the 5-methyl substituent from the acyl group of the side chain; and
omission of the acyl group of the side chain.

5. A process according to claim 4, wherein the compound obtained has a 4(S)-methylhexanoyl group at the end of the side chain.

6. A process according to any of the preceding claims, wherein the coupling between the cyclic part and the side chain fragment is between D-Pro-9 and L-Orn-8.

7. A process according to any of claims 1 to 5, wherein the coupling between the cyclic part and the side chain fragment is between L-Orn-8 and D-allo-Ile-7.

8. A process according to any of claims 1 to 5, wherein the coupling between the cyclic part and the side chain fragment is between D-Val-10 and D-Pro-9.

9. A process according to any of claims 1 to 5, wherein the coupling between the cyclic part and the side chain fragment is between D-allo-Ile-7 and D-allo-Thr-6.

10. A process according to any of claims 1 to 5, wherein the coupling between the cyclic part and the side chain fragment is between L-Val-11 and D-Val-10.
